(19) United States
(12) Patent Application Publication
(10) Pub. No.: US 2024/0425909 A1
(43) Pub. Date: Dec. 26, 2024

(54) COMPOSITIONS, KITS, AND METHODS FOR QUANTIFICATION OF NUCLEIC ACID SEQUENCES USING AN INTERNAL QUANTITATIVE STANDARD

(71) Applicant: LIFE TECHNOLOGIES CORPORATION, Carlsbad, CA (US)

(72) Inventors: Say Li KONG, Singapore (SG); San Min LEOW, Singapore (SG); Andy YING, Fremont, CA (US); Manohar R. FURTADO, San Ramon, CA (US); Grace Yu Hui WONG, Singapore (SG); Daryn KENNY, Pleasant Hill, CA (US); Jacelyn Hong Yi PHUA, Singapore (SG); Xiaohan XU, Singapore (SG)

(21) Appl. No.: 18/701,789

(22) PCT Filed: Oct. 20, 2022
(86) PCT No.: PCT/US2022/047259

§ 371 Date: Apr. 16, 2024

Related U.S. Application Data
(60) Provisional application No. 63/257,718, filed on Oct. 20, 2021.

(51) Int. Cl. C12Q 1/6851; C12Q 1/70

(52) U.S. Cl. CPC C12Q 1/6851; C12Q 1/701; C12Q 1/703; C12Q 2600/158; C12Q 2600/166

(57) ABSTRACT

Disclosed are compositions, kits, and methods for amplifying and quantifying a target nucleic acid from a sample. Compositions, kits, and methods enable the quantification of a target nucleic acid from a sample using an internal quantification standard disposed in the same reaction volume as the target nucleic acid, thereby eliminating the need for additional amplification reactions and processing to generate a standard curve. Compositions, kits, and methods also enable the comparison of target nucleic acid loads between two or more test samples by normalizing measured levels of the target nucleic acid in each sample according to relative levels of endogenous nucleic acid in each test sample.

### CROSS-REFERENCE TO RELATED APPLICATIONS

This application claims priority to and the benefit of U.S. Provisional Patent Application No. 63/257,718, filed on Oct. 20, 2021, which is incorporated herein by reference in its entirety.

### FIELD

The present teachings relate to compositions, kits, and methods for quantifying a target nucleic acid from a sample. In particular, embodiments described herein enable quantification of a target nucleic acid using an internal quantitative standard (IQS) within the same reaction volume as the target nucleic acid sequence.

### BACKGROUND

Assays to detect target nucleic acid sequences of interest are widely used in molecular biology and medicine, particularly in polymerase chain reaction (PCR) processes. Clinical applications typically involve collection of a sample from a subject, extraction of nucleic acid, and subjecting the extracted sample to amplification conditions in the presence of target-specific primers. The presence of amplification products thus indicates that the target nucleic acid was present within the sample, whereas failure to measure amplification products indicates that the target nucleic acid was absent or was present in levels too low for detection. Such assays are therefore useful for detecting and monitoring pathogenic diseases, among other applications such as analysis of environmental samples.

Standard quantitative polymerase chain reaction (qPCR) processes measure the amplification of target DNA by monitoring a target-specific fluorescent signal associated with amplification of the target sequence. The output is a quantification cycle (Cq) value that indicates the PCR cycle number at which the fluorescent signal corresponding to the target sequence passes the background fluorescence threshold level. Cq values are inversely proportional to the amount of target nucleic acid in the sample. That is, lower Cq values indicate higher amounts of the target nucleic acid whereas higher Cq values indicate lower amounts of target nucleic acid (or issues with the PCR process). Cq values do not, however, directly indicate the actual concentration of target nucleic acid in the sample.

Determining a quantity of the target nucleic acid in a test sample is sometimes achieved by generating a standard curve that correlates Cq values to target nucleic acid concentrations. A standard curve is generated by amplifying a series of known concentrations of the target nucleic acid and measuring the resulting Cq values. Although beneficial in some applications, generating a standard curve requires that several standalone reactions be carried out. This decreases throughput because additional reaction volumes (e.g., wells in a well plate) must be dedicated to standard curve samples, as well as requiring additional operator time and additional equipment and reagent usage. Moreover, standard curves cannot readily account for “well to well” (i.e., reaction volume specific) variability between reactions. Further, because standard curves are typically generated by forming serial dilutions of the known target sequence, the standard curve may inherently incorporate dilution errors introduced during the serial dilution process.

Accordingly, there is an ongoing need for systems, methods, kits, and other embodiments that quantify a target nucleic acid from a sample.

### BRIEF DESCRIPTION OF THE DRAWINGS

FIGS. 1A and 1B illustrate methods for quantifying a target nucleic acid from a sample.

FIG. 2 schematically compares an example competitive IQS and an example non-competitive IQS.

FIGS. 3A-3B illustrate amplification plots showing possible relationships between target nucleic acid Cq values and internal quantitative standard Cq values.

FIGS. 4A and 4B illustrate the effects of utilizing a correction factor in determining a quantity of target nucleic acid.

FIGS. 5A and 5B illustrate results of a SARS-COV-2 assay comparing target quantitation using a standard curve and target quantitation using an IQS according to the present disclosure.

FIG. 5C illustrates Cq values for an IQS and a viral target using multiple test compositions of different viral target concentrations, the results showing that the IQS provided a substantially constant Cq value unaffected by differences in target concentrations.

### DETAILED DESCRIPTION

All publications and patent applications cited herein, as well as the Appendices attached hereto, are incorporated by reference in their entirety for all purposes to the same extent as if each individual Appendix, publication or patent application were specifically and individually indicated to be so incorporated by reference. Although the Appendices attached hereto may include particular examples that reference specific target nucleic acids, formulations, and process steps, it will be understood that these examples may be modified by using any of the formulations, components, and/or process steps described elsewhere herein.

When an example “embodiment” or a particular “assay” is described herein, it will be understood that the features of the embodiment may be applicable to a composition (e.g., the particular physical components of an assay such as primers and/or probes), a kit (e.g., primers and/or probes and additional buffers, reagents, etc.), or a method (e.g., a process for detecting and/or quantifying target nucleic acids) as appropriate. For simplicity, many embodiments are presented by describing “assays”, but it will be understood that the associated methods of using the assays are also intended to form part of this disclosure.

### Overview of Compositions, Kits, and Methods for Quantifying a Target Nucleic Acid

Embodiments described herein are directed to compositions, kits, and methods for amplifying and quantifying a target nucleic acid using an IQS disposed in the same reaction volume as the target. The IQS may also be synonymously referred to herein as a control nucleic acid, an internal reference, or simply as a control.

Use of an IQS beneficially minimizes or eliminates the need for a standard curve to achieve quantification of the target nucleic acid. The use of an IQS can beneficially increase throughput because reaction volumes (e.g., wells) no longer need to be utilized for standard curve samples. Similarly, the use of an IQS can minimize reagent use and overall processing requirements. Moreover, as compared to a standard curve, an IQS better normalizes inter reaction volume variables (i.e., differences from well to well). For example, in reverse transcription PCR (RT-PCR) applications, an IQS can provide better normalization against variables such as RNA integrity and reverse transcription efficiencies.

In one embodiment, a method for quantifying a target nucleic acid in a test sample comprises: (a) providing a test sample comprising a target nucleic acid in a reaction volume; (b) providing a known amount of an IQS to the same reaction volume; (c) amplifying at least a portion of the target nucleic acid by subjecting the reaction volume to amplification conditions in the presence of target-specific primers; (d) amplifying at least a portion of the IQS by subjecting the reaction volume to amplification conditions in the presence of IQS-specific primers; (e) determining Cq values for the target nucleic acid and the IQS; (f) determining a target-to-IQS relative quantification ratio (RQ) value based on the Cq values of the target nucleic acid and the IQS; (g) optionally, determining a correction factor (CF) based on a difference between amplification efficiency of the target nucleic acid and the IQS; and (h) determining a quantity of the target nucleic acid in the test sample based on the known amount of the IQS and the target-to-IQS RQ value, optionally as modified by the CF.

In some embodiments, a method further comprises: amplifying an endogenous nucleic acid (e.g., RNase P) in the test sample by subjecting the test sample to amplification conditions in the presence of endogenous sequence primers; and normalizing the determined quantity of target nucleic acid based on relative level of endogenous nucleic acid in the test sample. For example, a method may include determining a Cq value for the endogenous nucleic acid; determining an endogenous-to-IQS RQ value based on the Cq values of the endogenous nucleic acid and the IQS; determining a quantity of the endogenous nucleic acid in the test sample based on the known amount of the IQS and the endogenous-to-IQS RQ value; and using the determined quantity of endogenous nucleic acid to normalize the determined quantity of the target nucleic acid.

In some embodiments, amplification of the target nucleic acid, amplification of the IQS, and/or amplification of the endogenous nucleic acid sequence happen simultaneously. In some embodiments, the method is utilized in an RT-PCR application. For example, the step of amplifying at least a portion of the target nucleic acid and/or the step of amplifying at least a portion of the IQS can include a reverse transcription reaction.

The IQS may be mixed with or otherwise contacted with a “raw” sample (i.e., a sample that has not undergone a nucleic acid extraction process). In this manner, the IQS can function as a process control as well as an amplification reaction control. That is, because the IQS is also subject to the same processing steps (e.g., extraction processes) as the other nucleic acids of the sample, it functions as an indicator of the effects of the processing steps. For example, failure to measure any of the control and/or measuring significantly lower levels of the control than expected, can indicate to the user that there may be issues with the processing and/or amplification reaction steps of a particular reaction run. Use of the IQS in this manner also helps to normalize variables that can arise due to extraction and/or other processing steps. Alternatively, the IQS may be added following nucleic acid extraction of the sample.

In embodiments where the IQS is added to the sample prior to processing steps such as nucleic acid extraction, the IQS may be armored. Examples of armor for the IQS include bacteriophage MS2 coat protein and others known in the art. In some embodiments, the IQS comprises or is derived from a mammalian RNA virus vector with bacteriophage MS2 vector for the armored RNA. As an example, the RNA insert size may be up to 200-300 bp, with armored RNA going up to about 3000 bp.

### Overview of Target Sequence Normalization Based on Endogenous Sequence Levels

In addition to quantification of a target sequence using an internal control, certain embodiments described herein are directed to normalization of the determined target sequence quantity based on levels of an endogenous sequence. Such normalization enables better comparisons between samples where the levels of target nucleic acid can be affected by sample collection and/or sample processing variables, which can lead to inconsistencies in levels of organic matter from sample to sample.

Inconsistencies in levels of organic matter from sample to sample make it difficult to generate meaningful comparisons of the total load of target nucleic acid between samples, even for samples from a single subject. In many applications it would be useful to determine how the load of target nucleic acid in a subject is changing over time, such as for monitoring the progression of a pathogenic disease. In the current state of the art, however, meaningful comparisons of this type cannot be made due to excessive variation between samples. For example, where testing of two different samples results in measured absolute quantities of target nucleic acid, it cannot be concluded that one sample actually demonstrates lower or higher levels of the target nucleic acid compared to the other, as any differences (or even coincidental similarities) may actually be caused by differences in the amount or concentration of organic matter collected in the samples.

Described herein according to some embodiments are compositions, kits, and methods configured to quantify target nucleic acids and to enable meaningful comparisons between multiple test samples by normalizing measured levels of the target nucleic acid in each sample according to relative levels of endogenous nucleic acid in each test sample. Embodiments described herein beneficially enable improved diagnosis and/or monitoring of disease progression in a subject over time, allowing medical professionals to better determine whether target nucleic acid associated with the pathogen is increasing, decreasing, or staying the same within the subject over time. This information can improve disease diagnosis, treatment, and/or prognosis by better illustrating treatment effects, highlighting risk thresholds, monitoring efficacy of therapeutic agents or processes, and/or indicating outcome probabilities, for example.

### Target Nucleic Acids

As disclosed herein, the target nucleic acid may be viral, bacterial, fungal, or eukaryotic. The target nucleic acid may be from a pathogen. In embodiments related to pathogenic disease diagnosis and/or monitoring, the target pathogen can be any pathogen that leaves detectable levels of nucleic acid within the subject because of infection of the subject. The pathogen may be a virus, bacteria, fungus, or eukaryotic parasite. In some embodiments, the target nucleic acid associated with the pathogen is obtainable through saliva collection and/or through a swab-based collection process. Swab-based collection processes commonly involve nasopharyngeal or oropharyngeal swabs, though certain embodiments are applicable to other types of swab samples, such as cheek swabs, wound swabs, skin swabs, aural swabs, anal swabs, vaginal swabs, or swabs of other anatomical locations. In some embodiments, the target nucleic acid associated with the pathogen is obtainable through a blood collection process. In some embodiments, the whole blood or part thereof (e.g., plasma) can be used to provide target nucleic acids. In some embodiments, blood samples may be collected using blood collection substrates (e.g., dried blood spots may be collected on suitable filter paper). In some embodiments, blood may be drawn directly from a subject. For example, heel “sticks” taking small amounts of blood may be utilized as a sample collection method. Blood may additionally or alternatively be drawn from other appropriate anatomical locations (e.g., finger).

In some embodiments, the target nucleic acid is associated with a virus. In some embodiments, the virus in an RNA virus. In some embodiments, the target nucleic acid is associated with a human immunodeficiency virus (HIV), including HIV-1 and HIV-2. Suitable nucleic acid targets within the HIV genome include the gag, LTR, integrase, or pol regions of the HIV genome, or others known to those of skill in the art.

Examples of respiratory microorganisms that may be targeted are listed in Table 1, below. Assays targeting one or more of the below organisms (and/or other organisms) include appropriate primers to enable amplification of target nucleic acid sequences associated with the pathogens, and optionally one or more probes to aid in detection of amplification products that target one or more newly emerging pathogens or microorganisms of interest.

TABLE 1
Example Respiratory Microorganisms

| | | | | |
|---|---|---|---|---|
| AdV 1 | FluB (pan) | CoV HKU1 | Parecho V | M. catarrhalis |
| HBoV | hPIV1 | CoV NL63 | Bordetella spp. | M. pneumoniae |
| HHV3 | hPIV2 | CoV OC43 | B. holmesii | S. aureus |
| HHV4 | hPIV3 | Mumps | B. pertussis | S. pneumoniae |
| HHV5 | hPIV4 | MERS-CoV | C. pneumoniae | P. jirovecii |
| HHV6 | RSVA | SARS-CoV | H. influenzae | M. tuberculosis |
| FluA (pan) | RSVB | SARS-CoV-2 | K. pneumoniae | Aspergillus |
| FluA (H1) | hMPV | Entero V (pan) | L. pneumophila | |
| FluA (H3) | Measles | Entero V D68 | | |
| | CoV 229E | Rhino V 1of2 | | |
| | | Rhino V 2of2 | | |

For a selected pathogen or other target organism, one or more nucleic acids may be targeted by designing target-specific primers that enable amplification of the target nucleic acid when the sample and the target-specific primers are subjected to amplification conditions. One of skill in the art is equipped to design appropriate primers. Such methods are described in Basu, Chhandak (Ed.) “PCR Primer Design” (Methods in Molecular Biology) 2^{nd} edition (2015).

Although not limited to any particular pathogen or set of pathogens, certain embodiments are configured for quantifying target nucleic acids associated with coronaviruses, in particular the SARS-COV-2. The SARS-COV-2 virus, also known as 2019-nCOV, is associated with the human respiratory disease COVID-19. The virus isolated from early cases of COVID-19 was provisionally named 2019-nCOV. The Coronavirus Study Group of the International Committee on Taxonomy of Viruses has subsequently given the official designation of SARS-COV-2. For the purposes of this disclosure SARS-COV-2 and 2019-nCOV are considered to refer to the same virus.

The genetic sequence of the initially characterized “reference” form of SARS-CoV-2 is based on the sequence associated with NCBI accession no. NC_045512.2 (see GenBank: MN908947.3) which describes a genome of 29,903 base pairs. Because SARS-CoV-2 is an RNA virus, it can mutate with relatively high frequency, which has led to the emergence of new variants, and it is likely that additional variants will continue to emerge over time.

In some embodiments, potential target genes for SARS-COV-2 include the Orf1a, Orf1b, S, E, M, and N genes, among several other accessory proteins. The SARS-CoV-2 genome encodes two large genes Orf1a and Orf1b, which encode 16 non-structural proteins (NSP1-NSP16). These NSPs are processed to form a replication-transcription complex (RTC) that is involved in genome transcription and replication. The structural genes encode the structural proteins, spike(S), envelope (E), membrane (M), and nucleocapsid (N). The accessory proteins are unique to SARS-COV-2 in terms of number, genomic organization, sequence, and function.

Table 2 illustrates some of the mutations that have occurred in the SARS-COV-2 genome, as well as some of their associated variants, where known. The numbering system used to designate these mutations is based on the “reference” sequence as defined above. For example, the mutation “S.N501Y.AAT.TAT” refers to a mutant form of the spike(S) protein wherein amino acid residue no. 501 is changed from asparagine (A) to tyrosine (Y). The latter portion of the label “AAT.TAT” compares the reference codon to the mutant codon, and in this example illustrates that the mutation is associated with a change from an adenine (A) to a thymine (T) (i.e., the AAT of the reference codon is changed to a TAT in the mutant codon). Note that RNA comprises uracil (U), but notation included herein may sometimes simply refer to the corresponding DNA base pair thymine (T). The initial part of the label specific to the protein involved and/or the latter portion of the label specific to the nucleotide mutation may occasionally be dropped from the label for convenience. The latter portion of the label may also be shortened to simply show the single reference nucleotide and mutant nucleotide, rather than the entire reference and mutant codon. Those with skill in the art will readily recognize the mutation nomenclature used herein.

TABLE 2
SARS-CoV-2 Mutations

| | | | Earliest Documented |
|---|---|---|---|
| Mutation | WHO Label | Associated Variants | Samples |
| S.D215G.GAT.GGT | Beta | B.1.351 | South Africa |
| S.D614G.GAT.GGT | Various | B.1.1.207, P.1, | Nigeria, United |
| | | B.1.1.33, B.1.1.7, | Kingdom, South |
| | | B.1.177, B.1.258, | Africa, Brazil/ |
| | | B.1.351, B.1.525, | Amazon |
| | | Mink variant | |
| S.delH69V70 | Alpha | B.1.1.7, B.1.258, | United Kingdom |
| | | B.1.525 | |
| S.delY144 | Alpha | B.1.1.7 | United Kingdom |
| S.E484K.GAA.AAA | Beta, | P.1, B.1.1.33, | South Africa, Brazil |
| | Gamma, | B.1.351, B.1.525 | |
| | Eta | | |
| S.E484Q.GAA.CAA | Delta | B.1.617, B.1.617.1, | India |
| | (absence | B.1.617.3 | |
| | of this | | |
| | mutation) | | |
| S.F888L.TTT.CTT | Eta | B.1.525 | 20A/S:484K United |
| | | | Kingdom |
| S.K417N.AAG.AAT | Beta | B.1.351 | South Africa |
| S.K417T.AAG.ACG | Gamma | P.1 | Brazil/Amazon |
| S.L18F.CTT.TTT | Gamma | P.1, B.1.351 | South Africa, |
| | | | Brazil/Amazon |
| S.L452R.CTG.CGG | Delta, | B.1.617, B.1.617.1, | India, California |
| | Epsilon | B.1.617.2, B.1.617.3, | |
| | | B.1.429 | |
| S.N439K.AAC.AAA | | B.1.258 | |
| S.N501Y.AAT.TAT | Alpha, | P.1, B.1.1.7, | United Kingdom, South |
| | Beta, | B.1.351 | African, Brazil/ |
| | Gamma | | Amazon |
| S.P681H.CCT.CAT | Alpha | B.1.1.207, B.1.1.7 | Nigeria, United |
| | | | Kingdom |
| S.P681R.CCT.CGT | Delta | B.1.617.1, B.1.617.2, | India |
| | | B.1.617.3 | |
| S.S13I.AGT.ATT | Epsilon | B.1.427, B.1.429 | California |
| S.S477N.AGC.AAC | Lota | B.1.526 | New York |
| S.T20N.ACC.AAC | Gamma | P.1 | Brazil/Amazon |
| S.V1176F.GTT.TTT | | P.2 | Brazil |
| Orf1ab.A13057T | Mu | B.1.621 | Colombia |
| Orf1.A1708D.GCT.GAT | Alpha | B.1.1.7 | United Kingdom |
| S.A222V.GCT.GTT | | B.1.177 | |
| CoV1.A28272T | Mu | B.1.621 | Colombia |
| S.A570D.GCT.GAT | Alpha | B.1.1.7 | United Kingdom |
| S.A701V.GCA.GTA | Beta | B.1.351 | South Africa |
| N.D3L.GAT.CTA | | | |
| S.D80A.GAT.GCT | Beta | B.1.351 | South Africa |
| S.E583D.GAG.GAT | | B.177 | |
| S.E583D.GAG.GAC | | B.177 | |
| S.EFR156-158G | Delta | B.1.617.2 | India |
| S.F157L.TTC.TTA | | A.23 | |
| S.F157S.TTC_TCC | Iota | B.1.526 | United States |
| S.G446V.GGT.GTT | | | |
| S.G769V.GGA.GTA | | | |
| S.H66D.CAT.GAT | | | |
| ORF1.K1795Q.AAA.CAA | | | |
| S.L452Q.CTG.CAG | Lambda | C.37 | Mink |
| S.N450K.AAT.AAG | | B.1.214 | |
| S.N460K.AAT.AAA | | | |
| S.N460K.AAT.AAG | | | |
| S.N460T.AAT.ACT | | | |
| P5743S.CCA_TCA | Mu | B.1.621 | Colombia |
| orf8.Q27ST.CAA.TAA | Alpha | B.1.1.7 | United Kingdom |
| S.Q498R.CAA.CGA | | | |
| S.Q52R.CAG.CGG | Eta | 1.525 | |
| S.Q613H.CAG.CAT | | A.23 | |
| S.Q677H.CAG.CAT | Eta | 1.525 | |
| S.Q677H.CAG.CAC | Eta | 1.525 | |
| S.R2461.AGA.ATA | Beta | B.1.351 | South Africa |
| S.R346K.AGA_AAA | Mu | B.1.621 | Colombia |
| S.S982A.TCA.GCA | Alpha | B.1.1.7 | United Kingdom |
| T1055A.ACA_GCA | Mu | B.1.621 | Colombia |
| S.T478K.ACA.AAA | Delta | B.1.617.2 | |
| S.T716I.ACA.ATA | Alpha | B.1.1.7 | United Kingdom |
| S.T95I.ACT_ATT | Iota, Mu | B.1.526, B.1.621.1 | United States, |
| | | | Colombia |
| S.V367F.GTC.TTC | | A.23 | |
| S.W152L.TGG.TTG | | R.1 | |
| S.Y453F.TAT.TTT | | B.1.1.298 | Mink |
| S.D1118H.G_C | | B.1.1.7 | United Kingdom |

Certain embodiments are directed to quantifying a target SARS-COV-2 nucleic acid. In some embodiments, one or more target-specific primers are targeted to a SARS-CoV-2 nucleic acid corresponding to reference SARS-COV-2. In some embodiments, one or more target-specific primers are targeted to a SARS-COV-2 nucleic acid corresponding to an existing or future variant form of SARS-COV-2. Embodiments may target one (e.g., in a singleplex reaction) or more (e.g., in a multiplex reaction) nucleic acid sequences associated with any of the SARS-COV-2 genes described herein. Typically, embodiments target one or more nucleic acid sequences associated with the Orf1a, Orf1b, S, or N gene.

Exemplary assays suitable for targeting SARS-COV-2 include TaqCheck™ SARS-COV-2 Fast PCR Assay Kit (Thermo Fisher Scientific, Catalog No. A47693), TaqPath™ COVID-19 Combo Kit (Thermo Fisher Scientific, Catalog No. A47813), TaqPath™ COVID-19 Combo Kit Advanced (Thermo Fisher Scientific, Catalog No. A47814), TaqMan™ SARS-COV-2 with RNase P Assay 2.0 (Thermo Fisher Scientific, Catalog No. A51121), TaqMan™ SARS-COV-2 RNase P Assay Kit (Thermo Fisher Scientific, Catalog No. A49564), CoviPath™ COVID-19 RT PCR Kit (Thermo Fisher Scientific, Catalog Nos. A50780 and A52000), and TaqMan™ SARS-COV-2 Fast PCR Combo Kit 2.0 (Thermo Fisher Scientific, Catalog No. A51607).

Other Exemplary assays/kits that may be utilized in conjunction with the components, kits, and methods described herein include TaqMan™ Drug Metabolism Genotyping Assay (Thermo Fisher Scientific Catalog number: 4362691), TaqMan™ Gene Expression Assay (FAM) (Thermo Fisher Scientific Catalog number: 4351368). Custom TaqMan™ Gene Expression Assay, FAM (Thermo Fisher Scientific Catalog number: 4331348), TaqMan™ SNP Genotyping Assay, human (Thermo Fisher Scientific Catalog number: 4351379), VetMAX™-Gold AIV Detection Kit (Thermo Fisher Scientific Catalog number: 4485261), VetMAX™-Gold Trich Detection Kit (Thermo Fisher Scientific Catalog number: 4483869), VetMAX™ PRRSV NA & EU Controls (Thermo Fisher Scientific Catalog number: 4405548).

### Methods for Quantifying a Target Nucleic Acid Using an Internal Quantitative Standard

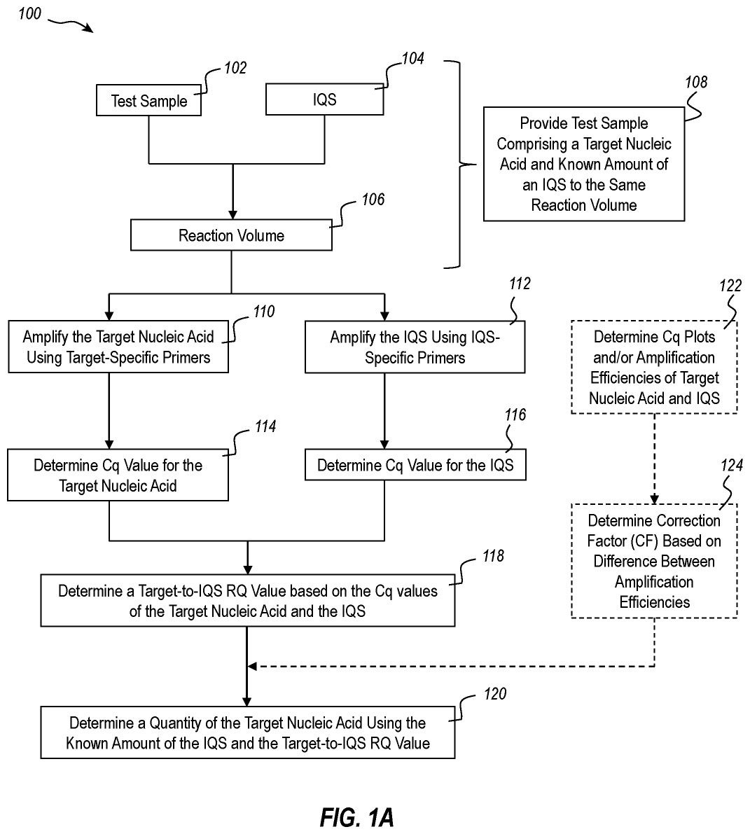

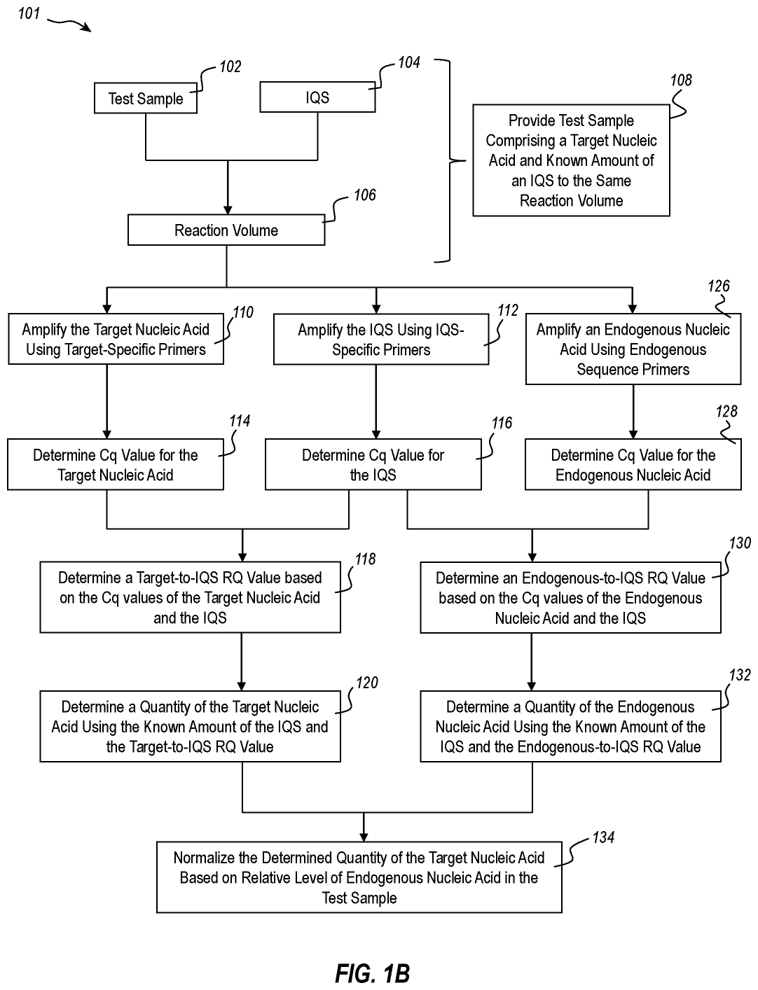

FIG. 1A illustrates a method 100 for quantifying a target nucleic acid using an internal control. The method provides the test sample 102 and a known amount of the IQS 104 to the same reaction volume 106 (step 108). As disclosed herein, the target nucleic acid may be viral, bacterial, fungal, or eukaryotic. The target nucleic acid may be from a pathogen. The target nucleic acid may be from a respiratory pathogen. In some embodiments, the target nucleic acid is from SARS-COV-2. In some embodiments, the target nucleic acid is an HIV sequence. Some embodiments quantify a single target nucleic acid (e.g., a singleplex reaction), whereas other embodiments quantify multiple target nucleic acids (e.g., a multiplex reaction).

In some embodiments, the IQS comprises an exogenous nucleic acid. In some embodiments, the IQS comprises a synthetic nucleic acid, such as a “xeno” sequence designed to be different from any known biological sequence. As described in more detail below, such IQS sequences may be utilized as a “non-competitive” standard that does not compete with the target nucleic acid for primers in the amplification reaction. In some embodiments, the IQS is designed to comprise one or more portions that match the target nucleic acid. As described in more detail below, such IQS sequences may be utilized as a “competitive” standard that does compete with the target nucleic acid for primers in the amplification reaction. Although a competitive standard competes with the target nucleic acid, the beneficial tradeoff is that the IQS and target sequences are more similar and therefore more likely to behave similarly during amplification, which can be beneficial with respect to subsequent quantification calculations.

The method 100 also includes the steps of amplifying the target nucleic acid using target-specific primers (step 110) and amplifying the IQS using IQS-specific primers (step 112). These steps may be performed simultaneously or sequentially in any order. In some embodiments, amplification is carried out via PCR. In some embodiments, amplification of the target nucleic acid and/or the IQS comprises a reverse transcription reaction. Additional details regarding amplification are provided elsewhere herein, and it will be understood that those details are applicable to the method 100. In some embodiments, the target nucleic acid is a SARS-COV-2 nucleic acid, and the target-specific primers are specific to one or more of the Orf1a gene, the Orf1b gene, the N gene, or the S gene of SARS-COV-2. In some embodiments, the target nucleic acid is a HIV nucleic acid, and the target-specific primers are specific to one or more of the gag, LTR, integrase, or pol regions of HIV, for example.

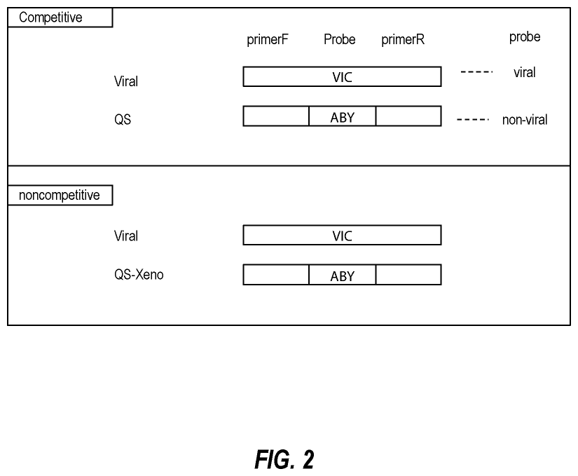

In some embodiments, the target-specific primers and the IQS-specific primers are different. In some embodiments, the target-specific primers and the IQS-specific primers have at least one primer in common. FIG. 2 schematically illustrates the difference between a competitive and a non-competitive arrangement. In the illustrated competitive configuration, the target sequence (“viral”) and the IQS sequence (“QS”) are amplified by the same set of primers. However, the IQS includes a portion that hybridizes with a “non-viral” probe that has a different sequence and different label (e.g., ABY rather than VIC) to enable differentiation from the target sequence. In some embodiments, the target-specific probe and the IQS-specific probe are similar other than a small region (e.g., 3-8 nucleotides, or about 5 nucleotides) to enable differential hybridization. In some embodiments, the target-specific probe and the IQS-specific probe have sequence regions that share about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotides. In the illustrated noncompetitive configuration, the primers, probes, and dye channels are different between the target nucleic acid and the control nucleic acid.

Referring to FIG. 1A, the method 100 also includes the steps of determining the Cq value for the target nucleic acid (step 114) and determining the Cq value for the IQS (step 116). Once the Cq values have been determined, the method determines a target-to-IQS RQ value based on the Cq values of the target nucleic acid and the IQS (step 118). The target-to-IQS RQ value represents a ratio of the target sequence to the control sequence in the test sample.

In some embodiments, the RQ value is determined according to Formula I:

$${RQ}_{Target - to - IQS} = {2}^{- ( Δ Cq )} = {2}^{- ( Target Cq - IQS Cq )} = \frac{{2}^{- ( Target Cq )}}{{2}^{- ( IQS Cq )}}$$

where “Target Cq” is the Cq value of the target nucleic acid and “IQS Cq” is the Cq value of the control nucleic acid. This formula assumes 100% amplification efficiency for the target and control sequences, which is sufficient for many applications.

In some embodiments, the RQ value is determined according to Formula II:

$${RQ}_{Target - to - IQS} = \frac{{( 1 + Target Efficiency )}^{- ( Target Cq )}}{{( 1 + IQS Efficiency )}^{- ( IQS Cq )}}$$

where “Target Efficiency” is the amplification efficiency of the target nucleic acid (with 1 being 100% efficiency) and “IQS Efficiency” is the amplification efficiency of the IQS (with 1 being 100% efficiency). This formula can be utilized where 100% efficiency is not a suitable assumption and/or where a more granular determination is desired.

The method 100 also includes the step of determining a quantity of the target nucleic acid using the known amount of the IQS and the target-to-IQS RQ value (step 120). The quantity of the target nucleic acid (Target qty) may be determined according to Formula III:

Target qty=IQS qty×RQ_{Target-to-IQS}

wherein “IQS qty” is the known amount of the IQS.

In some embodiments, the quantity of the target nucleic acid is also determined using a correction factor. As show, the method 100 optionally includes the steps of determining Cq plots (e.g., including determining the slopes of the Cq plots) and/or the amplification efficiencies of the target nucleic acid and the IQS (step 122) and determining the correction factor based on differences between the Cq plot information and/or amplification efficiencies (step 124).

For example, the correction factor may be calculated by: determining a Cq value for the target nucleic acid for at least one known concentration; determining a Cq value for the IQS for the same at least one known concentration; and defining the correction factor as a ratio between the respective Cq values. In some embodiments, the correction factor is determined by measuring multiple Cq values for the target and IQS at multiple known concentrations. In some embodiments, the correction factor is determined by averaging the Cq value ratios across the multiple known concentrations.

When a correction factor is utilized, the quantity of the target nucleic acid (Target qty) may be determined according to Formula IV:

Target qty=IQS qty×RQ_{Target-to-IQS}×CF

where “CF” is the correction factor and RQ is the target-to-IQS RQ. The correction factor thus mitigates the effects of inherent measurement bias due to differences in amplification characteristics between the target sequence and IQS.

FIG. 1B illustrates an embodiment of a related method 101 that incorporates amplification of an endogenous sequence and normalization of the determined quantity of target nucleic acid relative to the determined quantity of the endogenous sequence. In the method 101, several steps are the same as in method 100 and the following description will therefore describe the additional steps.

As shown, the method 101 includes the step of amplifying an endogenous nucleic acid using endogenous sequence primers (step 126). This amplification may be performed simultaneously with steps 110 and/or 112 or sequentially with steps 110 and/or 112 in any order. The method 101 also includes determining the Cq value for the endogenous nucleic acid (step 128).

Endogenous nucleic acid may be present in organic matter (e.g., cells and/or extracellular material such as mucous or cellular debris) of test sample and is therefore expected to be present in amounts proportional to the amount of organic matter in the sample. That is, detection of the target and the endogenous control may be impacted by amount of biomass of specimen. Preferable endogenous nucleic acids are stably expressed across test samples and minimally affected by test conditions, extraction processes, and subject differences. Some embodiments utilize protein-coding endogenous nucleic acids such as beta-actin or glyceraldehyde 3-phosphate dehydrogenase (GAPDH). More preferred embodiments typically utilize sequences that code for ribosomal RNA molecules rather than proteins. For example, in some preferred embodiments, the endogenous nucleic acid is an RNase P sequence. Other examples include: 18S ribosomal RNA; peptidylprolyl isomerase A (cyclophilin A); ribosomal protein L13a; ribosomal protein large P0; beta-2-microglobulin; tyrosine 3-monooxygenase/tryptophan 5-monooxygenase activation protein, zeta polypeptide; succinate dehydrogenase; transferrin receptor (p90, CD71); aminolevulinate, delta-, synthase 1; glucuronidase, beta; hydroxymethyl-bilane synthase; hypoxanthine phosphoribosyltransferase 1; TATA box binding protein; and tubulin, beta polypeptide.

The method 101 also includes the step of determining an endogenous-to-IQS RQ value based on the Cq values of the endogenous nucleic acid and the IQS (step 130). This step is similar to step 118 but provides a ratio of the endogenous sequence to the IQS rather than a ratio of the target sequence to the IQS.

As shown in FIG. 1B, the IQS used to determine the endogenous-to-IQS RQ value may be the same as that used to determine the target-to-IQS RQ value. Alternative embodiments utilize a second internal control. For example, in some embodiments a first IQS is utilized to determine the target-to-IQS RQ value while a second IQS is utilized to determine the endogenous-to-IQS RQ value.

The method 101 also includes the step of determining a quantity of the endogenous nucleic acid using the known amount of the IQS and the endogenous-to-IQS RQ value (step 132). The quantity of the endogenous nucleic acid (Endogenous qty) may be determined according to Formula V:

Endogenous qty=IQS qty×RQ_{Endogenous-to-IQS}

wherein “Control qty” is the known amount of control nucleic acid and RQ is the endogenous-to-control RQ.

As in method 100, the quantification steps of method 101 may also utilize one or more correction factors. The correction factor adjustment to the calculation of step 120, incorporating steps 122 and 124 as discussed above, may likewise be utilized in method 101. Moreover, although not shown in FIG. 1B for the sake of clarity, a similarly calculated correction factor may also be applied to determining the quantity of the endogenous sequence. Method 101 may include the steps of determining the amplification efficiency of the endogenous nucleic acid and the amplification efficiency of the IQS and determining the correction factor based on differences between these amplification efficiencies.

For example, the correction factor may be calculated by: determining a Cq value for the endogenous nucleic acid for at least one known concentration; determining a Cq value for the IQS for the same at least one known concentration; and defining the correction factor as a ratio between the respective Cq values. In some embodiments, the correction factor is determined by measuring multiple Cq values for the endogenous nucleic acid and the IQS at multiple known concentrations. In some embodiments, the correction factor is determined by averaging the Cq value ratios across the multiple known concentrations.

When a correction factor is utilized, the quantity of the endogenous nucleic acid (Endogenous qty) may be determined according to Formula VI:

Endogenous qty=IQS qty×RQ_{Endogenous-to-IQS}×CF

where “CF” is the correction factor and RQ is the endogenous-to-IQS RQ. The correction factor thus mitigates the effects of inherent measurement bias due to differences in amplification characteristics between the endogenous sequence and control sequence.

The method 101 also includes the step of normalizing the determined quantity of the target nucleic acid relative to the determined level of the endogenous nucleic acid in the test sample (step 134).

In preferred embodiments, the Cq plot slopes and/or amplification efficiencies of the target nucleic acid and the IQS are substantially similar. In other preferred embodiments, the Cq plot slopes and/or amplification efficiencies of the target nucleic acid and the endogenous nucleic acid are substantially similar. In other words, the target nucleic acid and the IQS preferably have comparable linearity in their respective Cq plots, and the target nucleic acid and the endogenous control preferably have comparable linearity in their respective Cq plots.

As used herein, the term “comparable linearity in Cq plot” (and similar terms) indicates that the separate Cq plots have slopes that are similar enough to enable effective quantitation of the target nucleic acid using the embodiments disclosed herein. For example, in some applications, slopes that differ by no more than 10% have comparable linearity in Cq plot. Other threshold values (e.g., slopes that differ by no more than 50%, 45%, 40%, 35%, 30%, 25%, 20%, 15%, 10%, 9%, 8%, 7%, 6%, 5%, 4%, 3%, 2%, or 1%) may be applicable in other circumstances. “Comparable linearity in Cq plot” may also be determined according to Cq values. For example, comparable linearity in Cq plot may be determined where the Cq value of the IQS differs from the Cq value of the target by no more than 10%, or by no more than another appropriate threshold (e.g., Cq values that differ by no more than 50%, 45%, 40%, 35%, 30%, 25%, 20%, 15%, 10%, 9%, 8%, 7%, 6%, 5%, 4%, 3%, 2%, or 1%). The IQS is preferably provided at a concentration that is optimized to minimize variability in Cq value as affected by the presence or amount of target nucleic acid. That is, the IQS is preferably provided at a concentration that provides a predictable Cq value that is substantially unaffected by the presence of the target nucleic acid. The IQS will have a linear range defined as the acceptable range of Cq values.

Although similarity in the slope of Cq plots is preferred, disclosed embodiments may also be utilized in circumstances where Cq plot slopes are dissimilar. Such embodiments may utilize a correction factor as described herein, for example. Use of a correction factor becomes more preferable as differences increase between Cq plot slopes of the target, IQS, and/or endogenous sequence.

In some embodiments, amplification of the target nucleic acid and amplification of the IQS and/or endogenous nucleic acid have efficiencies that differ by no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%. In some embodiments, a Cq plot of the target nucleic acid and a Cq plot of the IQS and/or endogenous nucleic acid have slopes (Cq/quantity) that differ by no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

### Additional Correction Factor Details

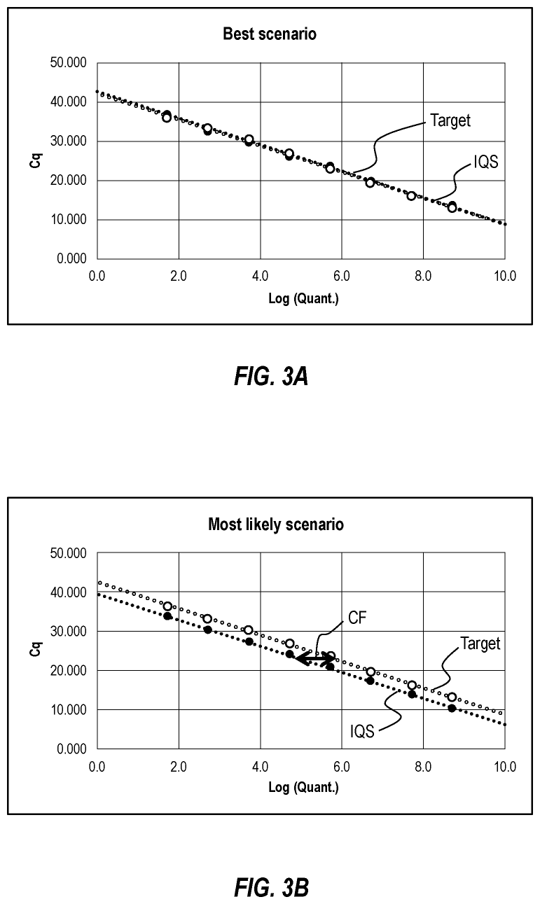

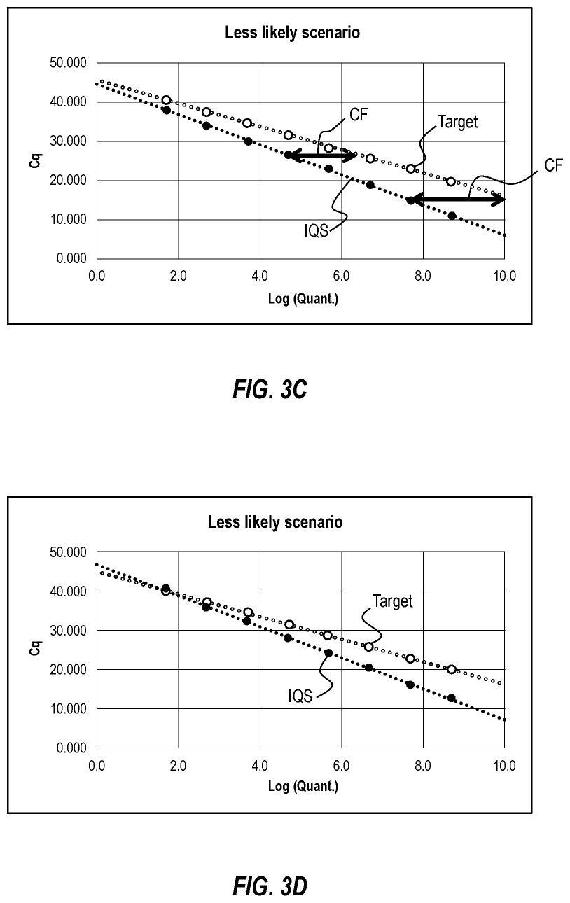

FIGS. 3A-3B illustrate example amplification plots showing Cq values of the target nucleic acid and the IQS and showing different scenarios that can factor into the correction factor determination. FIG. 3A illustrates the ideal scenario where the slope and intercept of the two curves are essentially the same. In this scenario, a correction factor is not needed because the substantially similar amplification efficiencies do not introduce systemic bias into the calculations.

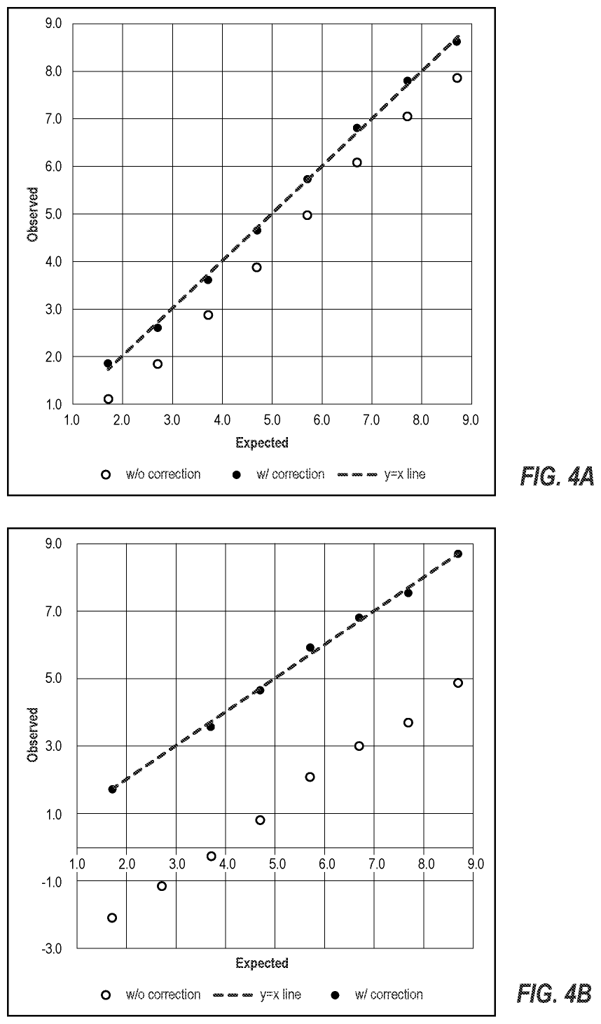

FIG. 3B illustrates the “most likely” scenario. Here, the target and control have substantially similar slopes, but different intercepts. In this scenario, the correction factor may be configured as a multiplier to correct for systemic bias resulting from the different intercepts of the amplification plot. In this scenario, the correction factor may be determined by providing known concentrations (e.g., serial dilutions) of target nucleic acid and the IQS and performing amplification (e.g., qPCR). Data points outside of the target nucleic acid or IQS assay linear range may be excluded. In one embodiment, RQ is calculated using Formula I and the initial target quantity is calculated using Formula III. A ratio of initial target quantity over the known target concentration is calculated for each dilution concentration. Each of these ratios represents a correction factor for the corresponding concentration or range of similar concentrations. Alternatively, in a simplified but still effective embodiment, the correction factor may be calculated as the average of these ratios over the linear range of the target nucleic acid and IQS. An example of determining a correction factor in such a scenario is described in greater detail below in Example 1 (with reference to Tables 4-5 and FIG. 4A).

FIG. 3C illustrates a scenario where the intercept is substantially the same, but the slopes are different. FIG. 3D illustrates a scenario where both the intercepts and the slopes are different. In these scenarios, as best shown in FIG. 3C, the correction can be adjusted depending on the Cq values rather than being applied as a constant multiplier. It is also preferable in such applications to use Formula II to calculate the RQ values. In this scenario, the correction factor may be determined by providing known concentrations (e.g., serial dilutions) of target nucleic acid and the IQS and performing amplification (e.g., qPCR). Data points outside of the target nucleic acid or IQS assay linear range may be excluded. In one embodiment, amplification efficiencies of the target nucleic acid and the IQS are determined using Formula VII:

$$Efficiency = 1 {0}^{- 1 / slope} - 1$$

where slope is determined from linear regression of the corresponding series of known concentrations (e.g., dilution series). RQ is calculated using Formula II and the initial target quantity is calculated using Formula III. A ratio of initial target quantity over the known target concentration is calculated for each dilution concentration. Each of these ratios represents a correction factor for the corresponding concentration or range of similar concentrations. Alternatively, in a simplified but still effective embodiment, the correction factor may be calculated as the average of these ratios over the linear range of the target nucleic acid and IQS. An example of determining a correction factor in such a scenario is described in greater detail below in Example 2 (with reference to Tables 6-7 and FIG. 4B).

### Sample Collection

The sample may be a veterinary sample, a clinical sample, a food sample, a forensic sample, an environmental sample (e.g., soil, dirt, garbage, sewage, air, or water), including food processing and manufacturing surfaces, or a biological sample. In some embodiments, the sample is a human sample. In some embodiments, the sample is a non-human sample. For instance, the sample may be from a non-human species such as a dog, cat, mink, livestock animals (e.g., pigs, cattle, sheep, goats), etcetera.

The sample can be collected by a healthcare professional in a healthcare setting, but in some instances, the sample may also be collected by the patient themselves or by an individual assisting the patient in self-collection. In some embodiments, the sample is a raw saliva sample collected-whether by self-collection or assisted/supervised collection—in a sterile tube or specifically-designed saliva collection device. The saliva collection tube/device may be a component of a self-collection kit having instructions for use, such as sample collection instructions, sample preparation or storage instructions, and/or shipping instructions. The raw saliva sample can be collected directly into a sealable container without any preservation solution or other fluid or substance in the container prior to receipt of the saliva sample within the container or as a result of closing/sealing the container.

In some embodiments, the nucleic acid fraction of the sample is extracted or purified from the sample—whether obtained via swab, from raw saliva, blood, or other bodily fluid—prior to any detection of target nucleic acids therein. The collection media is tested either directly or after extraction and purification of the nucleic acid target. In some embodiments, blood samples may be collected using blood collection substrates (e.g., dried blood spots may be collected on suitable filter paper). In some embodiments, blood may be drawn directly from a subject. For example, heel “sticks” taking small amounts of blood may be utilized as a sample collection method. Blood may additionally or alternatively be drawn from other appropriate anatomical locations (e.g., finger). The sample may include serum and/or plasma blood components.

Alternatively, certain embodiments can be adapted to detect target nucleic acid directly from a raw sample without a specific nucleic acid purification and/or extraction step prior to its use in downstream detection assays (e.g., RT-qPCR). In some embodiments, the sample is pre-treated prior to use. This can include, for example, heating the sample, such as by placing the raw sample on a heat block/water bath set to a heating temperature (e.g., about 95° C.) for a pre-treatment period (e.g., about 30 minutes), followed by combining the sample with a buffer or lysis solution. The buffer or lysis solution can include, for example, any nucleic-acid-amenable buffer such as TBE and may further include a detergent and/or emulsifier such as the polysorbate-type nonionic surfactant, Tween-20. The buffer or lysis solution may include a chaotropic agent and/or one or more enzymes, such as proteases, to help improve analyte detection by breaking down biological material and releasing analytes to make them more available for detection yet preserving nucleic acid targets.

A pre-heating step can provide many benefits, including, for example, breaking down mucus, making the sample more amenable to manipulation with laboratory equipment such as pipettes. The high heat can also cause thermal disruption of any prokaryotic and eukaryotic cells present in the sample and can also disrupt target organisms or virions present in the sample and thereby increase accessibility to any target nucleic acid.

The heat-treated sample may also be mixed (e.g., via vortexing the sample for at least 10 seconds) before and/or after equilibrating the heat-treated sample to room temperature. A lysis solution can then be prepared and combined (e.g., in 1:1 proportions) with the heat-treated sample to create a probative template solution for detecting the presence of target nucleic acid within the sample via nucleic acid amplification reactions (e.g., PCR, RT-PCR, qPCR, RT-qPCR, or the like). The lysis solution can include a nucleic-acid-amenable buffer such as TBE (and/or suitable alternative known in the art) combined with a detergent and/or emulsifier such as Tween-20, the polysorbate-type nonionic surfactant (and/or suitable alternative known in the art). The detergent and/or emulsifier can promote better mixing of the reagents and may also act to increase accessibility to any target nucleic acid within the sample (e.g., by removing lipid envelopes from virions).

It should be appreciated that in some embodiments, the disclosed compositions can include the sample mixed with a buffer and detergent/emulsifier. The sample can be added to a buffer/detergent mixture or vice versa. In some embodiments, the sample is combined with a buffer and then detergent is added to the saliva/buffer mixture. In other embodiments, the sample is directly combined with a buffer/detergent mixture. As a non-limiting example, a set of patient samples can be prepared as compositions for downstream analysis and detection of viral sequence by adding a volume of heat-treated sample for each patient into one (or a plurality) of wells in a multi-well plate. A volume of a buffer/detergent mixture (e.g., TBE+Tween-20) can then be added to each well containing a patient sample. Alternatively, a multi-well plate can be loaded with a volume of a buffer/detergent mixture into which a volume of heat-treated saliva is added. Once combined, this probative template solution can be used immediately or stored for later analysis. Such probative template solutions can also be combined with PCR reagents (e.g., buffers, dNTPs, master mixes, etc.) prior to or after storage.

### Nucleic Acid Amplification & Detection

Amplified products (“amplicons”) resulting from use of one or more embodiments described herein can be generated, detected, and/or analyzed using any suitable method and on any suitable platform. In some embodiments, the nucleic acid targets may be single-stranded, double-stranded, or any other nucleic acid molecule of any size or conformation. The nucleic acid assays described herein can include polymerase chain reaction (PCR) assays (see, e.g., U.S. Pat. No. 4,683,202), loop-mediated isothermal amplification (“LAMP”) (see, e.g., U.S. Pat. No. 6,410,278), and other methods described herein for detecting nucleic acid targets in a sample. In some embodiments, the PCR assays can be real time PCR or quantitative (qPCR) assays. In some embodiments, the PCR assays can be end point PCR assays.

In some embodiments, the primers described herein are used in nucleic acid assays at a concentration from about 100 nM to 1 mM (e.g., 300 nM, 400 nM, 500 nM, etc.), including intervening concentration amounts and ranges defined by endpoints selected from any two of the foregoing values. In some embodiments, probes described herein are also used in a nucleic acid assay and are provided at a concentration from about 50 nM to 500 nM (e.g., 75 nM, 125 nM, 250 nM, etc.), including intervening concentration amounts and ranges defined by endpoints selected from any two of the foregoing values.

The primers and/or probes described herein may further comprise a fluorescent or other detectable label. In some embodiments the primers and/or probes may further comprise a quencher and in other embodiments the probes may further comprise a minor groove binder (MGB) moiety. Suitable fluorescent labels include but are not limited to 6FAM, ABY, VIC, JUN, FAM. Suitable quenchers include but are not limited to QSY (e.g., QSY7 and QSY21), BHQ (Black Hole Quencher) and DFQ (Dark Fluorescent Quencher).

In some multiplex assay embodiments, various genomic regions are detected. Where SARS-COV-2 is a target, examples include assays configured to detect the Orf region (e.g., Orf1a, Orf1b, Orf1ab, Orf8), N protein region, S protein region, other genomic regions, and/or combinations thereof. Such multiplex assay embodiments may include multiple different probes for the same target genomic region in order to detect and/or distinguish between SARS-COV-2 variants. For example, a multiplex assay that includes a target in the S Protein genomic region may include multiple different probes (each differentially labelled) for different variant forms of the targeted S Protein genomic region. Other target regions (including the N Protein and/or Orf regions) may also include multiple probes corresponding to different variant forms of such target regions. Optionally, in some embodiments, control sequence primers and/or probes (e.g., JUN-labeled probes), such as for amplification and/or detection of bacteriophage MS2 or human RNase P control sequences, are included in the multiplex assays using suitable primer/probe sequences (and may be included as singleplex assays as well).

Where HIV is a target, an example multiplex assay may be configured to detect the gag region, LTR region, integrase region, pol region, other genomic regions, or combinations thereof. Such multiplex assay embodiments may include multiple different probes for the same target genomic region in order to detect and/or distinguish between HIV variants. For example, a multiplex assay that includes a target in the gag genomic region may include multiple different probes (each differentially labelled) for different variant forms of the targeted gag genomic region. Other target regions (e.g., the pol region) may also include multiple probes corresponding to different variant forms of such target regions. Optionally, in some embodiments, control sequence primers and/or probes, such as for amplification and/or detection of bacteriophage MS2 or human RNase P control sequences, are included in the multiplex assays using suitable primer/probe sequences (and may be included as singleplex assays as well).

Optionally, different qPCR assays can be plated into individual wells of a single array or multi-well plate, such as for example a TaqMan Array Card (see, e.g., Thermo Fisher Scientific, Waltham, MA; Catalog Nos. 4346800 and 4342265) or a MicroAmp multi-well (e.g., 96-well, 384-well) reaction plate (see, e.g., Thermo Fisher Scientific, Waltham, MA; Catalog Nos. 4346906, 4366932, 4306737, 4326659 and N8010560). Optionally, the different qPCR assays present in different wells of an array or plate can be dried or freeze-dried in situ and the array or plate can be stored or shipped prior to use. In some embodiments, the concepts described herein may be utilized in in situ hybridization applications not necessarily associated with PCR. For example, such applications include HER2/neu gene copy semi-quantitative detection in tissue sections, or RNA expression of analytes.

Primers and/or probes utilized in the disclosed methods need not have 100% homology to their targets to be effective, though in some embodiments, homology is substantially 100%. In some embodiments, primers and/or probes utilized herein have a homology to their respective target of about 50%, about 60%, about 70%, about 80%, about 85%, about 90%, about 95%, about 97%, about 98%, about 99%, about 99.9%, or up to substantially 100%. Some combinations of primers and/or probes may include primers and/or probes each with different homologies to their respective targets, and the homologies may be, for example, within a range with endpoints defined by any two of the foregoing values.

PCR and related methods are common methods of nucleic acid amplification. PCR is one, but not the only, example of a nucleic acid polymerase reaction method for amplifying a nucleic acid test sample comprising the use of a known nucleic acid as a primer and a nucleic acid polymerase to amplify or generate a specific target nucleic acid. In general, PCR utilizes a primer pair that consists of a forward primer and a reverse primer configured to amplify a target segment of a nucleic acid template. Typically, but not always, the forward primer hybridizes to the 5′ end of the target sequence and the reverse primer will be identical to a sequence present at the 3′ end of the target sequence. The reverse primer will typically hybridize to a complement of the target sequence, for example an extension product of the forward primer and/or vice versa. PCR methods are typically performed at multiple different temperatures, causing repeated temperature changes during the PCR reaction (“thermal cycling”). Other amplification methods, such as, e.g., loop-mediated isothermal amplification (“LAMP”), and other isothermal methods, such as those listed in Table 3, may require less or less extensive thermal cycling than does PCR, or require no thermal cycling. Such isothermal amplification methods are also contemplated for use with the assay compositions, kits, and methods described herein.

TABLE 3
Summary of optional isothermal amplification methods.

| | |
|---|---|
| NASBA | Nucleic acid sequence-based amplification (NASBA) is a method |
| | used to amplify RNA. |
| LAMP | Loop-mediated isothermal amplification (LAMP) is a single tube |
| | technique for the amplification of DNA. It typically uses 4-6 primers, |
| | which form loop structures to facilitate subsequent rounds of |
| | amplification. |
| HAD | Helicase-dependent amplification (HDA) uses the double-stranded |
| | DNA unwinding activity of a helicase to separate strands for in vitro DNA |
| | amplification at constant temperature. |
| RCA | Rolling circle amplification (RCA) starts from a circular DNA |
| | template and a short DNA or RNA primer to form a long single stranded |
| | molecule. |
| MDA | Multiple displacement amplification (MDA) is a technique that |
| | initiates when multiple random primers anneal to the DNA template and |
| | the polymerase amplifies DNA at constant temperature. |
| WGA | When MDA is used to amplify DNA from a whole genome of a |
| | cell it is called whole genome amplification (WGA). (Other methods of |
| | WGA include MALBAC, LIANTI, DOP-PCR.) |
| RPA | Recombinase polymerase amplification (RPA) is a low |
| | temperature DNA and RNA amplification technique. |

Methods of performing PCR are well known in the art; nevertheless, further discussion of PCR and other methods may be found, for example, in Molecular Cloning: A Laboratory Manual by Green and Sambrook, Cold Spring Harbor Laboratory Press, 4th Edition 2012, which is incorporated by reference herein in its entirety.

Certain embodiments target RNA viruses such as SARS-COV-2. SARS-COV-2 has a single-stranded positive-sense RNA genome. In some embodiments, therefore, the amplification reaction (e.g., LAMP or PCR) can be combined with a reverse transcription (RT) reaction, such as in RT-LAMP or RT-PCR, to convert the RNA genome to a cDNA template. The cDNA template is then used to create amplicons of the target sequences in the subsequent amplification reactions. In some embodiments, the RT-PCR may be a one-step procedure using one or more primers and one or more probes as described herein. In some embodiments, the RT-PCR may be carried out in a single reaction tube, reaction vessel (e.g., “single-tube” or “1-tube” or “single-vessel” reaction). In some embodiments, the RT-PCR may be carried out in a multi-site reaction vessel, such as a multi-well plate or array. In some embodiments, RT and PCR are performed in the same reaction vessel or reaction site, such as in 1-step or 1-tube RT-qPCR. Suitable exemplary RTs can include, for instance, a Moloney Murine Leukemia Virus (M-MLV) Reverse transcriptase, SuperScript Reverse Transcriptases (Thermo Fisher Scientific), SuperScript IV Reverse Transcriptases (Thermo Fisher Scientific), or Maxima Reverse Transcriptases (Thermo Fisher Scientific), or modified forms of any such RTs.

In some embodiments, different assay products (e.g., amplicons from different variants) can be independently detected or at least discriminated from each other. For example, different assay products may be distinguished optically (e.g., using optically different labels for each qPCR assay) or can be discriminated using some other suitable method, including as described in U.S. Patent Publication No. 2019/0002963, which is incorporated herein by reference in its entirety.

In some embodiments, the amplifying step can include performing qPCR, as that term is defined herein, qPCR is a sensitive and specific method for detecting and optionally quantifying amounts of starting nucleic acid template (e.g., coronaviral nucleic acid) in a sample. Methods of qPCR are well known in the art; one leading method involves the use of a specific hydrolysis probe in conjunction with a primer pair. The hydrolysis probe can include an optical label (e.g., fluorophore) at one end and a quencher that quenches the optical label at the other end. Other variations include an optical label and/or quencher positioned internally (i.e., not necessarily at the end) of the probe. Some probes may include more than one optical label and/or more than one quencher. In some embodiments, the label is at the 5′ end of the probe and cleavage of the 5′ label occurs via 5′ hydrolysis of the probe by the nucleic acid polymerase as it extends the forward primer towards the probe binding site within the target sequence. The separation of the probe label from the probe quencher via cleavage (or unfolding) of the probe results in an increase in optical signal which can be detected and optionally quantified. The optical signal can be monitored over time and analyzed to determine the relative or absolute amount of starting nucleic acid template present in the sample. Exemplary methods for polymerizing and/or amplifying and detecting nucleic acids suitable for use as described herein are commercially available as TaqMan assays (see, e.g., U.S. Pat. Nos. 4,889,818; 5,079,352; 5,210,015; 5,436, 134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084, 102; 6, 127, 155; 6,171, 785; 6,214,979; 6,258,569; 6,814,934; 6,821,727; 7,141,377; and/or 7,445,900, all of which are hereby incorporated herein by reference in their entirety).

TaqMan assays are typically carried out by performing nucleic acid amplification on a target polynucleotide using a nucleic acid polymerase having 5′-to-3′ nuclease activity, a primer capable of hybridizing to the target polynucleotide, and an oligonucleotide probe capable of hybridizing to said target polynucleotide 3′ relative to the primer. The oligonucleotide probe typically includes a detectable label (e.g., a fluorescent reporter molecule) and a quencher molecule capable of quenching the fluorescence of the reporter molecule. Typically, the detectable label and quencher molecule are part of a single probe. As amplification proceeds, the polymerase digests the probe to separate the detectable label from the quencher molecule. The detectable label is monitored during the reaction, where detection of the label corresponds to the occurrence of nucleic acid amplification (e.g., the higher the signal the greater the amount of amplification). Variations of TaqMan assays are known in the art and would be suitable for use in the methods described herein.

For example, a singleplex or multiplex qPCR can include a single TaqMan assay associated with a locus-specific sequence or multiple TaqMan assays respectively associated with a plurality of loci in a multiplex format. As a non-limiting example, a 4-plex reaction can include FAM (emission peak ˜517 nm), VIC (emission peak ˜551 nm), ABY (emission peak ˜580 nm), and JUN (emission peak ˜617 nm) dyes. In some embodiments, each dye is associated with one or more target sequences. In some embodiments, one or more dyes are quenched by a QSY quencher (e.g., QSY21). In some embodiments, each multiplex reaction allows up to 12 targets to be amplified and tracked real-time within a single reaction vessel. In some embodiments, up to 2, 4, 6, 8, 10, or 12 targets are amplified and tracked real-time within a single reaction vessel, using any combination of detectable labels disclosed herein or otherwise known to those of skill in the art. The aforementioned reporter dyes are optimized to work together with minimal spectral overlap for improved performance. Any combination of dyes described herein can additionally be combined with other dyes (e.g., Mustang Purple (emission peak ˜654 nm) or one or more Alexa Fluors (e.g., AF647 and AF676)), for use in monitoring fluorescence of a control or for use in a non-emission-spectrum-overlapping 5-plex assay. In addition, the QSY quencher is fully compatible with probes that have minor-groove binder quenchers.

Where multiple detection channels are utilized, it is desirable to minimize cross-talk between fluorescence reporters and select reporters that avoid excessive spectral overlap. One example of an assay that includes 5 detection channels incorporates the dyes FAM, ABY, VIC, and JUN, along with Mustang Purple (emission peak ˜654 nm) or an appropriate Alexa Fluor, for example. The dyes may be associated with a corresponding primer and/or with a probe of the assay, as described herein. Other embodiments may utilize other combinations of dyes to define different sets of detection channels (including in assays with more than 5 detection channels) according to particular preferences or application needs.

Detector probes may be associated with alternative quenchers, including without limitation, dark fluorescent quencher (DFQ), black hole quenchers (BHQ), Iowa Black, QSY quencher, and Dabsyl and Dabcel sulfonate/carboxylate Quenchers. Detector probes may also include two probes, wherein, for example, a fluorophore is associated with one probe and a quencher is associated with a complementary probe such that hybridization of the two probes on a target quenches the fluorescent signal or hybridization on the target alters the signal signature via a change in fluorescence. Detector probes may also include sulfonate derivatives of fluorescein dyes with SO₃ instead of the carboxylate group, phosphoramidite forms of fluorescein, phosphoramidite forms of Cy5.

It should be appreciated that when using more than one detectable label, particularly in a multiplex format, each detectable label preferably differs in its spectral properties from the other detectable labels used therewith such that the labels may be distinguished from each other, or such that together the detectable labels emit a signal that is not emitted by either detectable label alone. Exemplary detectable labels include, for instance, a fluorescent dye or fluorophore (e.g., a chemical group that can be excited by light to emit fluorescence or phosphorescence), “acceptor dyes” capable of quenching a fluorescent signal from a fluorescent donor dye, and the like, as described above. Suitable detectable labels may include, for example, fluoresceins (e.g., 5-carboxy-2,7-dichlorofluorescein; 5-Carboxyfluorescein (5-FAM); 5-Hydroxy Tryptamine (5-HAT); 6-JOE; 6-carboxyfluorescein (6-FAM); Mustang Purple, VIC, ABY, JUN; FITC; 6-carboxy-4′,5′-dichloro-2′, 7′-dimethoxy-fluorescein (JOE)); 6-carboxy-1,4-dichloro-2′,7′-dichloro fluorescein (TET); 6-carboxy-1,4-dichloro-2′,4′,5′,7′-tetra-chlorofluorescein (HEX); Alexa Fluor fluorophores (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), Cascade Blue, Cascade Yellow; Cy™ dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalama1), cyan fluorescent protein (e.g., ECFP, Cerulean, CyPet), yellow fluorescent protein (e.g., YFP, Citrine, Venus, YPet), FRET donor/acceptor pairs (e.g., fluorescein/fluorescein, fluorescein/tetramethylrhodamine, IAEDANS/fluorescein, EDANS/dabcyl, BODIPY FL/BODIPY FL, Fluorescein/QSY7 and QSY9), LysoTracker and LysoSensor (e.g., LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethyl-rhodamine), Tetramethylrhodamine (TRITC), WT), Texas Red, Texas Red-X, among others as would be known to those of skill in the art. In certain embodiments, the use of a ROX dye improves precision of quantitation by reducing well to well differences from master mix volume (and/or other contributors to imprecision) in the PCR detection plate.

Other detectable labels may be used in addition to or as an alternative to labelled probes. For example, primers can be labeled and used to both generate amplicons and to detect the presence (or concentration) of amplicons generated in the reaction, and such may be used in addition to or as an alternative to labeled probes described herein. As a further example, primers may be labeled and utilized as described in Nazarenko et al. (Nucleic Acids Res. 2002 May 1; 30 (9): e37), Hayashi et al. (Nucleic Acids Res. 1989 May 11; 17 (9): 3605), and/or Neilan et al. (Nucleic Acids Res. Vol. 25, Issue 14, 1 Jul. 1997, pp. 2938-39). Those of skill in the art will also understand and be capable of utilizing the PCR processes (and associated probe and primer design techniques) described in Zhu et al. (Biotechniques. 2020 July: 10.2144/btn-2020-0057).

Any of these systems and detectable labels, as well as many others, may be used to detect amplified target nucleic acids. In some embodiments, intercalating labels can be used such as ethidium bromide, SYBR Green I, SYBR GreenER, and PicoGreen (Life Technologies Corp., Carlsbad, CA), thereby allowing visualization in real-time, or end point, of an amplification product in the absence of a detector probe. In some embodiments, real-time visualization may include both an intercalating detector probe and a sequence-based detector probe. In some embodiments, the detector probe is at least partially quenched when not hybridized to a complementary sequence in the amplification reaction and is at least partially unquenched when hybridized to a complementary sequence in the amplification reaction. In some embodiments, probes may further comprise various modifications such as a minor groove binder to further provide desirable thermodynamic characteristics.

The labeled amplicon (or labeled derivative thereof) can be detected using any suitable method such as, for example, electrophoresis, hybridization-based detection (e.g., microarray, molecular beacons, and the like), chromatography, NMR, and the like. In one exemplary embodiment, the labeled amplicon is detected using capillary electrophoresis. In another embodiment, the labeled amplicon is detected using qPCR.

In some embodiments, the nucleic acid amplification assays as described herein are performed using a Real-time PCR (qPCR) instrument, including for example a QuantStudio Real-Time PCR system, such as the QuantStudio 5 RealTime PCR System (QS5), QuantStudio 7 RealTime PCR System (QS7), and/or QuantStudio 12K Flex System (QS12K), or a 7500 Real-Time PCR system, such as the 7500 Fast Dx system, from Thermo Fisher Scientific.

Some embodiments relate to kits containing primers and probes disclosed herein. Optionally, the kit can further include a master mix. In some embodiments, the master mix is TaqMan Fast Virus 1-Step Master Mix (Thermo Fisher Scientific, Catalog No. 44444432). In some embodiments, the master mix is TaqPath 1-Step RT-qPCR Master Mix, CG (Thermo Fisher Scientific, Catalog No. A15299). In other embodiments the master mix is TaqPath™ 1 Step Multiplex Master Mix (No ROX™) (Thermo Fisher Scientific, Catalog No. A48111, A28521). In some embodiments, the kit includes primers, probes and master mix sufficient to constitute a reaction mixture supporting amplification of one or more target regions from SARS-COV-2.

### Example Embodiments

The present disclosure includes, but is not limited to, embodiments represented by the following clauses:

Clause 1: A method for quantifying a target nucleic acid in a test sample, the method comprising: providing a test sample comprising a target nucleic acid in a reaction volume; providing a known amount of an internal quantitative standard (IQS) to the same reaction volume; amplifying at least a portion of the target nucleic acid by subjecting the reaction volume to amplification conditions in the presence of target-specific primers; amplifying at least a portion of the internal quantitative standard by subjecting the reaction volume to amplification conditions in the presence of IQS-specific primers; determining quantification cycle (Cq) values for the target nucleic acid and the IQS; determining a target-to-IQS relative quantification ratio (RQ) value based on the Cq values of the target nucleic acid and the IQS; optionally, determining a correction factor based on a difference between amplification efficiency of the target nucleic acid and the IQS; and determining a quantity of the target nucleic acid in the test sample based on the known amount of the IQS and the target-to-IQS RQ value, optionally as modified by the correction factor.

Clause 2: The method of clause 1, wherein the target nucleic acid is a viral nucleic acid.

Clause 3: The method of clause 2, wherein the target nucleic acid is a SARS-CoV-2 nucleic acid.

Clause 4: The method of clause 3, wherein the target-specific primers are specific to at least a portion of one or more of the Orf1a gene, the Orf1b gene, the N gene, or the S gene of SARS-COV-2.

Clause 5: The method of clause 2, wherein the target nucleic acid is from a human immunodeficiency virus (HIV).

Clause 6: The method of clause 5, wherein the target-specific primers are specific to at least a portion of one or more of the gag, LTR, integrase, or pol regions of HIV.

Clause 7: The method of any one of clauses 1-6, wherein the IQS is an exogenous nucleic acid.

Clause 8: The method of any one of clauses 1-7, wherein the IQS is a synthetic nucleic acid.

Clause 9: The method of any one of clauses 1-8, wherein the target-specific primers and the IQS-specific primers are different such that amplification of the target nucleic acid and amplification of the IQS are non-competitive.

Clause 10: The method of any one of clauses 1-8, wherein at least one of the target-specific primers is the same as at least one of the IQS-specific primers.

Clause 11: The method of clause 10, wherein the target-specific primers and the IQS-specific primers are the same.

Clause 12: The method of clause 10 or clause 11, further comprising using a target-specific probe configured to associate with amplicons of the target nucleic acid and an IQS-specific probe configured to associate with amplicons of the IQS, wherein the target-specific probe and the IQS-specific probe are different.

Clause 13: The method of clause 12, wherein the target-specific probe and the IQS-specific probe have different sequences.

Clause 14: The method of clause 13, wherein the target-specific probe and the IQS-specific probe have overlapping sequence regions that share about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases.

Clause 15: The method of any one of clauses 12-14, wherein the target-specific probe and the IQS-specific probe have different dye labels.

Clause 16: The method of any one of clauses 1-15, wherein the step of amplifying at least a portion of the target nucleic acid comprises a reverse transcription reaction.

Clause 17: The method of any one of clauses 1-16, wherein the step of amplifying at least a portion of the IQS comprises a reverse transcription reaction.

Clause 18: The method of any one of clauses 1-17, wherein the amplification of steps (c) and (d) are performed simultaneously.

Clause 19: The method of any one of clauses 1-18, wherein the method is performed without using a standard curve to determine the quantity of the target nucleic acid in the test sample.

Clause 20: The method of any one of clauses 1-19, wherein the method is performed without using a passive reference control dye.

Clause 21: The method of any one of clauses 1-20, wherein the RQ value is determined as:

$${2}^{- ( Δ Cq )} = {2}^{- ( Target Cq - IQS Cq )} = \frac{{2}^{- ( Target Cq )}}{{2}^{- ( IQS Cq )}} .$$

Clause 22: The method of any one of clauses 1-20, wherein the RQ value is determined as:

$$RQ = \frac{{( 1 + Target Efficiency )}^{- ( Target Cq )}}{{( 1 + IQS Efficiency )}^{- ( IQS Cq )}} .$$

Clause 23: The method of any one of clauses 1-22, wherein the correction factor is determined by: determining a Cq value for the target nucleic acid for at least one known concentration; determining a Cq value for the IQS for the same at least one known concentration; and defining the correction factor as a ratio between the respective Cq values.

Clause 24: The method of clause 23, wherein the correction factor is determined by measuring multiple Cq values for the target and IQS at multiple known concentrations.

Clause 25: The method of clause 24, wherein the correction factor is determined by averaging the Cq value ratios across the multiple known concentrations.

Clause 26: The method of any one of clauses 1-25, wherein the quantity of the target nucleic acid (Target qty) is determined by:

Target qty=IQS qty×RQ×CF

wherein “Control qty” is the known amount of IQS and “CF” is the correction factor.

Clause 27: The method of any one of clauses 1-26, further comprising extracting the target nucleic acid from a raw sample to form the test sample.

Clause 28: The method of clause 27, wherein the IQS is added to the raw sample prior to the extraction.

Clause 29: The method of clause 28, wherein the IQS is armored.

Clause 30: The method of clause 29, wherein the IQS is armored, optionally wherein the IQS is armored using MS2 coat protein and/or is a mammalian RNA virus vector with bacteriophage MS2 vector as armored RNA.

Clause 31: The method of any one of clauses 1-30, further comprising: amplifying an endogenous nucleic acid in the test sample by subjecting the test sample to amplification conditions in the presence of endogenous sequence primers; and normalizing the determined quantity of target nucleic acid based on relative level of endogenous nucleic acid in the test sample.

Clause 32: The method of clause 31, wherein the endogenous nucleic acid is RNase P and wherein the endogenous sequence primers are specific for RNase P.

Clause 33: The method of clause 31 or clause 32, further comprising: determining a Cq value for the endogenous nucleic acid; determining an endogenous-to-IQS RQ value based on the Cq values of the endogenous nucleic acid and the IQS; determining a quantity of the endogenous nucleic acid in the test sample based on the known amount of the IQS and the endogenous-to-IQS RQ value; and using the determined quantity of endogenous nucleic acid to normalize the determined quantity of the target nucleic acid.

Clause 34: The method of any one of clauses 31-33, wherein amplification of the target nucleic acid and amplification of the IQS and/or endogenous nucleic acid have efficiencies that differ by no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

Clause 35: The method of any one of clauses 31-34, wherein a Cq plot of amplification of the target nucleic acid and a Cq plot of amplification of the IQS and/or endogenous nucleic acid have slopes (Cq/quantity) that differ by no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

Clause 36: The method of clause 1, wherein the IQS comprises: a first sequence that is identical or complementary to a part of the target nucleic acid; and a second sequence that is different from or not complementary to the target nucleic acid.

Clause 37: The method of any one of clauses 1-36, wherein the test sample is a blood sample.

Clause 38: A composition for amplifying a target nucleic acid, the composition comprising: a DNA polymerase; a buffer; an internal quantitative standard (IQS); a pair of target-specific primers; and a pair of IQS-specific primers, wherein amplification of the IQS and amplification of the target nucleic acid have substantially similar efficiency.

Clause 39: The composition of clause 38, wherein the target-specific primers and the IQS-specific primers are different and thereby enable non-competitive amplification of the target nucleic acid and the IQS.

Clause 40: The composition of clause 39 for use in a method as in any one of clauses 1-9 or 16-37.

Clause 41: The composition of clause 38, wherein the target-specific primers and the IQS-specific primers have at least one primer in common to thereby enable competitive amplification of the target nucleic acid and the IQS.

Clause 42: The composition of clause 41 for use in a method as in any one of clauses 1-8 or 10-37.

Clause 43: The composition of clause 42, further comprising a target nucleic acid probe and a different, IQS probe.

Clause 44: The composition of clause 36, wherein the IQS comprises: a first sequence that is identical or complementary to a part of the target nucleic acid and a second sequence that is different from or not complementary to the target nucleic acid.

Clause 45: The composition of any one of clauses 38-44, wherein the composition further comprises a reverse transcriptase.

Clause 46: The composition of any one of clauses 38-45, wherein the IQS is an exogenous sequence.

Clause 47: The composition of any one of clauses 38-46, wherein the IQS is a synthetic sequence.

Clause 48: The composition of any one of clauses 38-47, wherein the target-specific primers are specific to a viral nucleic acid.

Clause 49: The composition of clause 48, wherein the target-specific primers are specific to a SARS-COV-2 nucleic acid.

Clause 50: The composition of clause 49, wherein the target-specific primers are specific to at least a portion of one or more of the Orf1a gene, the Orf1b gene, the N gene, or the S gene of SARS-COV-2.

Clause 51: The composition of clause 48, wherein the target-specific primers are specific to an HIV nucleic acid.

Clause 52: The composition of clause 51, wherein the target-specific primers are specific to at least a portion of one or more of the gag, LTR, integrase, or pol regions of HIV.

Clause 53: The composition of any one of clauses 38-52, wherein the IQS has a comparable linearity in a Cq plot to the target nucleic acid.

Clause 54: The composition of any one of clauses 38-53, further comprising a pair of endogenous sequence primers configured to enable amplification of an endogenous sequence.

Clause 55: The composition of clause 54, wherein the endogenous sequence primers are configured to enable amplification of RNase P.

Clause 56: The composition of any one of clauses 38-55, wherein amplification of the target nucleic acid and amplification of the IQS and/or endogenous nucleic acid have efficiencies that differ by no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

Clause 57: The composition of any one of clauses 38-56, wherein a Cq plot of amplification of the target nucleic acid and a Cq plot of amplification of the IQS and/or endogenous nucleic acid have slopes (Cq/quantity) that differ by no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

### EXAMPLES

The following Examples may reference specific target nucleic acids, compositions, formulations, and/or process steps. It will be understood, however, that these Examples may be modified by using any of the alternative components described elsewhere herein.

### Example 1: Correction Factor Determinations

Known quantities of a target nucleic acid and an IQS were provided in a qPCR reaction. The resulting amplification plots resembled the example shown in FIG. 3B. That is, the slopes were essentially the same but the intercepts were different. Values are shown below in Table 4.

TABLE 4
Amplification Plot Results

| | | Target |
|---|---|---|
| | IQS | Sequence |
| Slope | −3.348 | −3.352 |
| Intercept | 39.759 | 42.252 |
| r\hat{ }2 | 0.999 | 0.999 |
| Efficiency | 0.989 | 0.988 |

Table 5 shows the known values of target added, the measured Cq values of the IQS and the target, and the determined target quantities without the correction factor. As shown, the determined target quantities (without correction factor) systematically under-calculated the true amount as a result of the slight mismatch between the target and IQS amplification plots. In this example, the expected/observed values at each known concentration were averaged to provide a correction factor of 5.69. As shown in the last two columns on the right, multiplying the target quantities by the correction factor brought them back to or close to the expected values.

TABLE 5
Correction Factor Applied to Target Quantities

| Known Values of Target | | | | w/o correction factor | | | w correction factor | |
|---|---|---|---|---|---|---|---|---|
| | copies/mL | Measured Cq Values | | | log Target | expected/ | | log Target |
| copies/mL | (log) | IQS Cq | Target Cq | Target qty. | qty. | observed | Target qty. | qty. |
| 5.00E+08 | 8.7 | 10.405 | 13.204 | 7.18E+07 | 7.9 | 6.96 | 4.09E+08 | 8.6 |
| 5.00E+07 | 7.7 | 14.052 | 16.200 | 1.13E+07 | 7.1 | 4.43 | 6.42E+07 | 7.8 |
| 5.00E+06 | 6.7 | 17.439 | 19.499 | 1.20E+06 | 6.1 | 4.17 | 6.83E+06 | 6.8 |
| 5.00E+05 | 5.7 | 20.803 | 23.235 | 9.27E+04 | 5.0 | 5.40 | 5.28E+05 | 5.7 |
| 5.00E+04 | 4.7 | 24.201 | 26.891 | 7.75E+03 | 3.9 | 6.45 | 4.41E+04 | 4.6 |
| 5.00E+03 | 3.7 | 27.454 | 30.265 | 7.12E+02 | 2.9 | 7.02 | 4.06E+03 | 3.6 |
| 5.00E+02 | 2.7 | 30.232 | 33.067 | 7.01E+01 | 1.8 | 7.14 | 3.99E+02 | 2.6 |
| 5.00E+01 | 1.7 | 34.244 | 36.242 | 1.25E+01 | 1.1 | 3.99 | 7.13E+01 | 1.9 |
| | | | | | | Avg. = 5.69 | | |

This data is illustrated in graphical form in FIG. 4A. As shown, the dashed line represents where the observed values would be expected to fall. The series of quantities “w/o correction” were observed (y-axis values) at systematically lower values than expected (x-axis values). After application of the correction factor, the “w/correction” series shows that all measurements were brought back to where observed and expected values substantially align.

### Example 2: Additional Correction Factor Determinations

Known quantities of a target nucleic acid and an IQS were provided in a qPCR reaction. The resulting amplification plots resembled the example shown in FIG. 3C. That is, the slopes were different but the intercepts were similar. Values are shown below in Table 6.

TABLE 6
Amplification Plot Results

| | | Target |
|---|---|---|
| | IQS | Sequence |
| Slope | −3.958 | −2.904 |
| Intercept | 46.826 | 44.996 |
| r\hat{ }2 | 0.999 | 0.998 |
| Efficiency | 0.789 | 1.210 |

Table 7 shows the known values of target added, the measured Cq values of the IQS and the target, and the determined target quantities without the correction factor. As shown, the determined target quantities (without correction factor) systematically miscalculated the true amount as a result of the mismatch between the target and control amplification plots. In this example, the expected/observed values at each known concentration were averaged to provide a correction factor of 4771.07. As shown in the last columns to the right, multiplying the target quantities by the correction factor brought them back to or close to the expected values.

TABLE 7
Correction Factor Applied to Target Quantities

| Known Values of Target | | | | w/o correction factor | | | w correction factor | |
|---|---|---|---|---|---|---|---|---|
| | copies/mL | Measured Cq Values | | | log Target | expected/ | | log Target |
| copies/mL | (log) | IQS Cq | Target Cq | Target qty. | qty. | observed | Target qty. | qty. |
| 5.00E+08 | 8.7 | 12.637 | 19.861 | 1.13E+05 | 5.1 | 4438.07 | 5.38E+08 | 8.7 |
| 5.00E+07 | 7.7 | 16.131 | 22.456 | 1.10E+04 | 4.0 | 4550.89 | 5.24E+07 | 7.7 |
| 5.00E+06 | 6.7 | 20.644 | 25.342 | 1.54E+03 | 3.2 | 3249.02 | 7.34E+06 | 6.9 |
| 5.00E+05 | 5.7 | 23.940 | 28.569 | 8.10E+01 | 1.9 | 6170.84 | 3.87E+05 | 5.6 |
| 5.00E+04 | 4.7 | 27.920 | 31.685 | 6.94E+00 | 0.8 | 7209.15 | 3.31E+04 | 4.5 |
| 5.00E+03 | 3.7 | 32.454 | 34.414 | 1.11E+00 | 0.0 | 4489.14 | 5.31E+03 | 3.7 |
| 5.00E+02 | 2.7 | 35.866 | 36.617 | 1.41E−01 | −0.8 | 3538.18 | 6.74E+02 | 2.8 |
| 5.00E+01 | 1.7 | 40.404 | 40.256 | 1.11E−02 | −2.0 | 4523.27 | 5.27E+01 | 1.7 |
| | | | | | | Avg. = 4771.07 | | |

This data is illustrated in graphical form in FIG. 4B. As shown, the dashed line represents where the observed values would be expected to fall. The series of quantities “w/o correction” were observed (y-axis values) at systematically lower values than expected (x-axis values). After application of the correction factor, the “w/correction” series shows that all measurements were brought back to where observed and expected values substantially align.

### Example 3: Comparison of Quantification Using Standard Curve and Using Internal Control

Using SARS-COV-2 sequences as targets, assays were conducted to compare quantitation of the target using a standard curve and using an IQS. The SARS-COV-2 target was amplified using the TaqCheck™ SARS-COV-2 Fast PCR Assay (Thermo Fisher Scientific, Catalog No. A47693). The IQS was provided and amplified using the Xeno™ Internal Positive Control-LIZ™ Assay (Thermo Fisher Scientific, Catalog No. A29766).

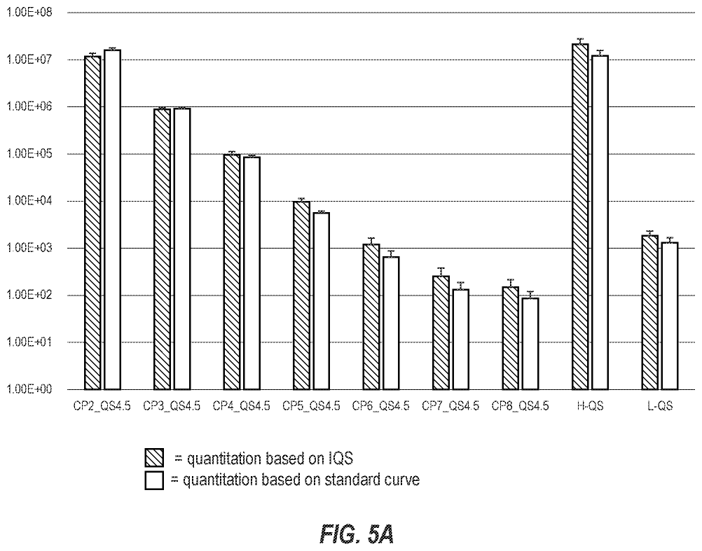

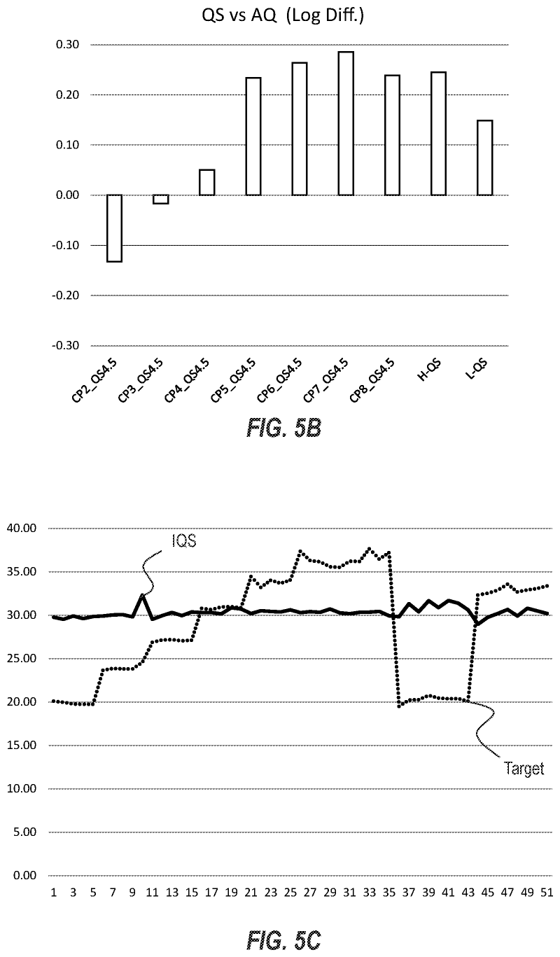

Results are illustrated in FIGS. 5A-5C. In FIG. 5A, the x-axis labels indicate various samples each of known concentration. The blue bars indicate quantitation results using an IQS according to the embodiments described herein, whereas the red bars indicate quantitation results based on use of a conventional standard curve. As shown, the IQS provided results similar to the standard curve. Similarly, FIG. 5B illustrates the Log difference of quantitation between the IQS-derived quantitation and the standard curve-derived quantitation. The x-axis labels represent samples of known concentration. The results of this testing illustrate that quantitation using an IQS, according to the embodiments disclosed herein, enable effective quantitation without the additional efforts and limitations associated with conventional standard curve-based methods.

In FIG. 5C, the y-axis values are Cq and the x-axis labels represent various samples. The blue series indicates Cq results for the IQS, whereas the red series indicates Cq results for the target. As shown, the IQS provided a substantially constant Cq value unaffected by differences in target concentrations.

1. A method for quantifying a target nucleic acid in a test sample, the method comprising:
(a) providing a test sample comprising a target nucleic acid in a reaction volume;
(b) providing a known amount of an internal quantitative standard (IQS) to the same reaction volume;
(c) amplifying at least a portion of the target nucleic acid by subjecting the reaction volume to amplification conditions in the presence of target-specific primers;
(d) amplifying at least a portion of the internal quantitative standard by subjecting the reaction volume to amplification conditions in the presence of IQS-specific primers;
(e) determining quantification cycle (Cq) values for the target nucleic acid and the IQS;
(f) determining a target-to-IQS relative quantification ratio (RQ) value based on the Cq values of the target nucleic acid and the IQS;
(g) optionally, determining a correction factor based on a difference between amplification efficiency of the target nucleic acid and the IQS; and
(h) determining a quantity of the target nucleic acid in the test sample based on the known amount of the IQS and the target-to-IQS RQ value, optionally as modified by the correction factor.

2. The method of claim 1, wherein the target nucleic acid is a viral nucleic acid.

3. The method of claim 2, wherein the target nucleic acid is a SARS-COV-2 nucleic acid.

4. The method of claim 3, wherein the target-specific primers are specific to at least a portion of one or more of the Orf1a gene, the Orf1b gene, the N gene, or the S gene of SARS-COV-2.

5. The method of claim 2, wherein the target nucleic acid is from a human immunodeficiency virus (HIV).

6. The method of claim 5, wherein the target-specific primers are specific to at least a portion of one or more of the gag, LTR, integrase, or pol regions of HIV.

7. The method of claim 1, wherein the IQS is an exogenous nucleic acid.

8. The method of claim 1, wherein the IQS is a synthetic nucleic acid.

9. The method of claim 1, wherein the target-specific primers and the IQS-specific primers are different such that amplification of the target nucleic acid and amplification of the IQS are non-competitive.

10. The method of claim 1, wherein at least one of the target-specific primers is the same as at least one of the IQS-specific primers.

11. The method of claim 10, wherein the target-specific primers and the IQS-specific primers are the same.

12. The method of claim 10, further comprising using a target-specific probe configured to associate with amplicons of the target nucleic acid and an IQS-specific probe configured to associate with amplicons of the IQS, wherein the target-specific probe and the IQS-specific probe are different.

13. The method of claim 12, wherein the target-specific probe and the IQS-specific probe have different sequences.

14. The method of claim 13, wherein the target-specific probe and the IQS-specific probe have overlapping sequence regions that share about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases.

15. The method of claim 12, wherein the target-specific probe and the IQS-specific probe have different dye labels.

16. The method of claim 1, wherein the step of amplifying at least a portion of the target nucleic acid comprises a reverse transcription reaction.

17. The method of claim 1, wherein the step of amplifying at least a portion of the IQS comprises a reverse transcription reaction.

18. The method of claim 1, wherein the amplification of steps (c) and (d) are performed simultaneously.

19. The method of claim 1, wherein the method is performed without using a standard curve to determine the quantity of the target nucleic acid in the test sample.

20. The method of claim 1, wherein the method is performed without using a passive reference control dye.

21. The method of claim 1, wherein the RQ value is determined as:
$${2}^{- ( Δ Cq )} = {2}^{- ( Target Cq - IQS Cq )} = \frac{{2}^{- ( Target Cq )}}{{2}^{- ( IQS Cq )}} .$$

22. The method of claim 1, wherein the RQ value is determined as:
$$RQ = \frac{{( 1 + Target Efficiency )}^{- ( Target Cq )}}{{( 1 + IQS Efficiency )}^{- ( IQS Cq )}} .$$

23. The method of claim 1, wherein the correction factor is determined by:
determining a Cq value for the target nucleic acid for at least one known concentration;
determining a Cq value for the IQS for the same at least one known concentration; and
defining the correction factor as a ratio between the respective Cq values.

24. The method of claim 23, wherein the correction factor is determined by measuring multiple Cq values for the target and IQS at multiple known concentrations.

25. The method of claim 24, wherein the correction factor is determined by averaging the Cq value ratios across the multiple known concentrations.

26. The method of claim 1, wherein the quantity of the target nucleic acid (Target qty) is determined by:
Target qty=IQS qty×RQ×CF
wherein “Control qty” is the known amount of IQS and “CF” is the correction factor.

27. The method of claim 1, further comprising extracting the target nucleic acid from a raw sample to form the test sample.

28. The method of claim 27, wherein the IQS is added to the raw sample prior to the extraction.

29. The method of claim 28, wherein the IQS is armored.

30. The method of claim 29, wherein the IQS is armored, optionally wherein the IQS is armored using MS2 coat protein and/or is a mammalian RNA virus vector with bacteriophage MS2 vector as armored RNA.

31. The method of claim 1, further comprising:
amplifying an endogenous nucleic acid in the test sample by subjecting the test sample to amplification conditions in the presence of endogenous sequence primers; and
normalizing the determined quantity of target nucleic acid based on relative level of endogenous nucleic acid in the test sample.

32. The method of claim 31, wherein the endogenous nucleic acid is RNase P and wherein the endogenous sequence primers are specific for RNase P.

33. The method of claim 31, further comprising:
determining a Cq value for the endogenous nucleic acid;
determining an endogenous-to-IQS RQ value based on the Cq values of the endogenous nucleic acid and the IQS;
determining a quantity of the endogenous nucleic acid in the test sample based on the known amount of the IQS and the endogenous-to-IQS RQ value; and
using the determined quantity of endogenous nucleic acid to normalize the determined quantity of the target nucleic acid.

34. The method of claim 31, wherein amplification of the target nucleic acid and amplification of the IQS and/or endogenous nucleic acid have efficiencies that differ by no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

35. The method of claim 31, wherein a Cq plot of amplification of the target nucleic acid and a Cq plot of amplification of the IQS and/or endogenous nucleic acid have slopes (Cq/quantity) that differ by no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

36. The method of claim 1, wherein the IQS comprises:
a first sequence that is identical or complementary to a part of the target nucleic acid; and
a second sequence that is different from or not complementary to the target nucleic acid.

37. The method of claim 1, wherein the test sample is a blood sample.

38. A composition for amplifying a target nucleic acid, the composition comprising:
a DNA polymerase;
a buffer;
an internal quantitative standard (IQS);
a pair of target-specific primers; and
a pair of IQS-specific primers,
wherein amplification of the IQS and amplification of the target nucleic acid have substantially similar efficiency.

39. The composition of claim 38, wherein the target-specific primers and the IQS-specific primers are different and thereby enable non-competitive amplification of the target nucleic acid and the IQS.

40. The composition of claim 39 for use in a method as in any one of claim 1-9 or 16-37.

41. The composition of claim 38, wherein the target-specific primers and the IQS-specific primers have at least one primer in common to thereby enable competitive amplification of the target nucleic acid and the IQS.

42. The composition of claim 41 for use in a method as in any one of claim 1-8 or 10-37.

43. The composition of claim 42, further comprising a target nucleic acid probe and a different, IQS probe.

44. The composition of claim 36, wherein the IQS comprises:
a first sequence that is identical or complementary to a part of the target nucleic acid and
a second sequence that is different from or not complementary to the target nucleic acid.

45. The composition of claim 38, wherein the composition further comprises a reverse transcriptase.

46. The composition of claim 38, wherein the IQS is an exogenous sequence.

47. The composition of claim 38, wherein the IQS is a synthetic sequence.

48. The composition of claim 38, wherein the target-specific primers are specific to a viral nucleic acid.

49. The composition of claim 48, wherein the target-specific primers are specific to a SARS-COV-2 nucleic acid.

50. The composition of claim 49, wherein the target-specific primers are specific to at least a portion of one or more of the Orf1a gene, the Orf1b gene, the N gene, or the S gene of SARS-COV-2.

51. The composition of claim 48, wherein the target-specific primers are specific to an HIV nucleic acid.

52. The composition of claim 51, wherein the target-specific primers are specific to at least a portion of one or more of the gag, LTR, integrase, or pol regions of HIV.

53. The composition of claim 38, wherein the IQS has a comparable linearity in a Cq plot to the target nucleic acid.

54. The composition of claim 38, further comprising a pair of endogenous sequence primers configured to enable amplification of an endogenous sequence.

55. The composition of claim 54, wherein the endogenous sequence primers are configured to enable amplification of RNase P.

56. The composition of claim 38, wherein amplification of the target nucleic acid and amplification of the IQS and/or endogenous nucleic acid have efficiencies that differ by no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

57. The composition of claim 38, wherein a Cq plot of amplification of the target nucleic acid and a Cq plot of amplification of the IQS and/or endogenous nucleic acid have slopes (Cq/quantity) that differ by no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.
